(19) United States
(12) Patent Application Publication
(10) Pub. No.: US 2007/0059840 A1
(43) Pub. Date: Mar. 15, 2007

(54) Hemostasis Analysis Device and Method

(75) Inventors: Eli Cohen, Skokie, IL (US); Vincent J. Contini, Powell, OH (US); Peter Delmenico, Chicago, IL (US); Matthew S. Fleming, Columbus, OH (US); Thomas D. Haubert, Columbus, OH (US); Gabriel Raviv, Glenview, IL (US); Ronnie Raviv, Chicago, IL (US); Mark R. Wilson, Columbus, OH (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: HAEMOSCOPE CORPORATION, Niles, IL (US)

(21) Appl. No.: 11/383,567

(22) Filed: May 16, 2006

Related U.S. Application Data
(60) Provisional application No. 60/681,375, filed on May 16, 2005.

(51) Int. Cl. G01N 33/86

(52) U.S. Cl. 436/69

(57) ABSTRACT

Apparatus for measuring hemostasis includes a container for holding a sample to be tested and a bobber configured to be buoyantly suspended on the sample. A magnet is secured to the bobber. A drive assembly is coupled to the container for driving the container in an oscillating motion. A magnetic field generator is disposed adjacent the container and configured to generate a magnetic field in the vicinity of the magnet. A magnetic field strength detector is disposed adjacent the container, and the magnetic field strength detector is configured to sense changes in the magnetic field as a result of movement of the bobber and magnet responsive to the oscillating motion of the container and clotting of the sample.

### CROSS-REFERENCE TO RELATED APPLICATIONS

This patent claims benefit under 35 U.S.C. § 119(e) to U.S. Provisional Application Ser. No. 60/681,375, filed May 16, 2005 entitled Hemostasis Analsysis Device and Method, the disclosure of which is hereby expressly incorporated herein for all purposes.

### TECHNICAL FIELD

This patent relates to a device and method for the measurement of hemostasis.

### BACKGROUND

Devices for measuring hemostasis are known, for example, from the commonly assigned U.S. Pat. No. 6,225,126. These devices, which take whole blood as the sample, provide comprehensive measurement and data of the hemostasis characteristics of the sample. Analysis of the results provides an ability to diagnose and treat numerous hemostasis disorders.

### BRIEF DESCRIPTION OF THE DRAWINGS

FIG. 1 is a front schematic illustration of a hemostasis analysis device in accordance with one of the described embodiments.

FIG. 2 is a plan schematic illustration of the hemostasis analysis device shown in FIG. 1.

### DETAILED DESCRIPTION

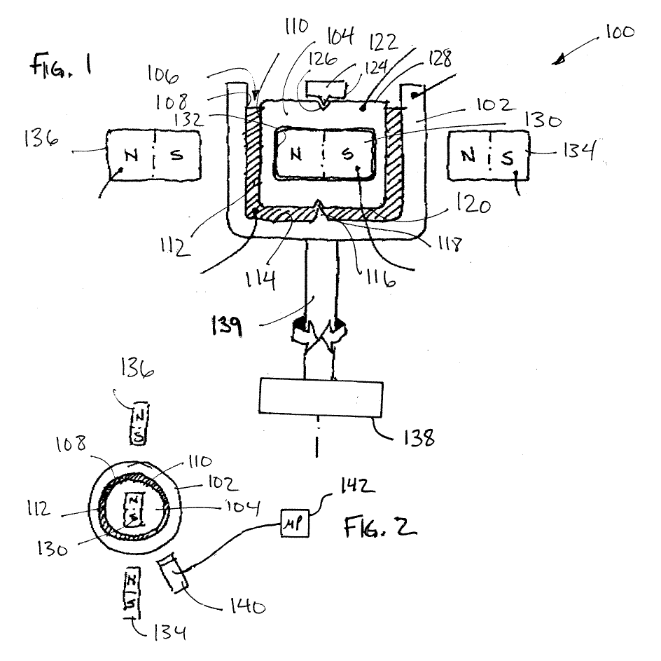

An apparatus for measuring hemostasis 100 uses a container 102 for a blood sample into which a bobber 104 is buoyantly suspended. The container 102 and the bobber 104 are formed as cylinders. A space 106 is defined between an inner wall 108 of the container 102 and an outer surface 110 of the bobber 104, into which a blood sample 112 to be tested is disposed and on which the bobber 104 floats. As shown in FIG. 1, a bottom surface 114 of the container 102 includes a pintle 116, and the bobber 104 is formed with a corresponding dimple 118 on a bottom surface 120 thereof. The pintle 116 and dimple 118 cooperate to center the bobber 104 within the container 102. An optional locating member 122, supported from a suitable structure of the apparatus 100 (not depicted) or from the container 102 itself, may be provided and includes a pintle 124 that engages a dimple 126 formed in a top surface 128 of the bobber 104, also for ensuring the bobber 104 remains centered in the container 102. An alternative arrangement may place the pintle on the bobber and form the container and/or the support structure to include a corresponding dimple. In this regard, the arrangement of the locating members, i.e., the pintles/dimples, is flexible and can accommodate various design requirements.

The container 102 is formed of suitable medical grade plastic, as is the bobber 104. Alternatively, the container 102 and/or the bobber 104 may be formed of any suitable, non-magnetic material including non-magnetic metal, composite materials, glasses, ceramics and the like.

The bobber 104 is further formed with a cavity 132 into which a magnet 130 is disposed. For example, the bobber 104 may be molded around the magnet 130 encapsulating it therein. Fixed to the structure of the device (not depicted) externally of the container 102 are first and second fixed magnets 134 and 136, arranged to have a north pole and a south pole, respectively, disposed toward the container 102. The first and second fixed magnets 134 and 136 present a magnetic field that acts to align the bobber 104, via interaction with the magnet 130 as it floats within the container 102. The magnetic field further provides a predetermined resistance to rotation of the bobber 104. The magnetic field may be generated by other structures, however, including by electric coils or other structures capable of generating a magnetic field.

The container 102 is further adapted to couple to an oscillating drive assembly 138. As depicted, the container 102 is formed with a shaft 139 that engages source of driving energy. The drive assembly 138 may be a cam and motor arrangement, although a direct motor drive, a gear drive or any other suitable drive may be used. The container 102 and bobber 104 are configured to be replaceably inserted into the apparatus 100. In this regard, the container 102 and the bobber 104 are intended to be a one use only, disposable item, and will generally be sold as an assembly.

A magnetic field strength detector 140, also secured to the structure (not depicted) of the apparatus 100 is positioned adjacent the container 102 at a suitable radial location. In the embodiment depicted in FIG. 1, the magnetic field strength detector 140 is positioned adjacent to but offset from the first fixed magnet 134. The magnetic field strength detector 140 may be positioned anywhere relative to the container 102 and the bobber 104 such that it is effective to sense changes in magnetic field responsive to movement of the bobber 104 and the magnet 130 therein. In an exemplary embodiment, the magnetic field sensor may be positioned in the same x-y plane as the fixed magnets 134 and 136 but along a perpendicular axis. In such an arrangement, the magnetic field detector 140 and the magnets 134 and 136 form a “T.” Furthermore, the container 102, the bobber 104, the magnets 134 and 136 and the magnetic field detector 140 may all be coplanar.

The drive assembly 138 is controlled to rotate the container 102 repeatedly through a small angular range, for example of about 3 to about 10 degrees. Initially, with no clotting of the sample, the magnetic field of the first and second fixed magnets 134 and 136 is sufficient to resist rotational movement of the bobber 104. As the blood clots, linking between the container 102 and the bobber 104 occurs until the clotting blood and linking between the container 102 and the bobber 104 is sufficiently strong to overcome the magnetic field causing movement of the bobber 104 with the container 102. Gradually, as blood clot strength increases, the rotation of the bobber 104 becomes aligned and the bobber 104 moves substantially in unison with the container 102 or with only a slight lag. As lysis begins, the clot strength decreases, and the movement of the bobber 104 is again more substantially influenced by the magnetic field of the first and second fixed magnets 134 and 136, until the bobber 104 no longer moves substantially in unison with the container 102. Eventually, the strength of the clotting blood will decrease to the point that the strength of the magnetic field will again hold the bobber 104 against rotational movement.

The magnetic field strength detector 140 is operable to sense displacement of the bobber 104 from its aligned position by sensing variations of the magnetic field. As will be appreciated, as the bobber 104 rotates, movement of the magnet 130 changes the magnetic field in the vicinity of the magnetic field detector 140. The magnitude of the changes correspond to the magnitude of the displacement of the bobber 104, which, as discussed above, is related to the strength of the clotting blood sample.

The magnetic field strength detector may be coupled to a processor, computer or other suitable device 142 to receive the magnetic field strength data. From the magnetic field strength data, the device 142 is configured to determine various hemostasis parameters, such as time to initial clot formation, rate of clot strengthening, maximum clot strength and the lysis time, as are well known. It will be appreciated that the strength of the magnetic field presented by the first and second fixed magnets 134 and 136 should be such that the strength of the clotting blood does not completely overcome the field, as it would not be possible to detect maximum clot strength. However, the field strength should not be so great that the time to initial clot formation is not observable.

While the present disclosure is susceptible to various modifications and alternative forms, certain embodiments are shown by way of example in the drawings and the herein described embodiments. It will be understood, however, that this disclosure is not intended to limit the invention to the particular forms described, but to the contrary, the invention is intended to cover all modifications, alternatives, and equivalents defined by the appended claims.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term by limited, by implication or otherwise, to that single meaning. Unless a claim element is defined by reciting the word “means” and a function without the recital of any structure, it is not intended that the scope of any claim element be interpreted based on the application of 35 U.S.C. §112, sixth paragraph.

We claim:

1. Apparatus for measuring hemostasis comprising:
a container for holding a sample to be tested;
a bobber configured to be buoyantly suspended on or within the sample and a magnet secured to the bobber;
a drive assembly coupled to the container for driving the container in an oscillating motion;
a magnetic field generator disposed adjacent the container and configured to generate a magnetic field in the vicinity of the magnet; and
a magnetic field strength detector disposed adjacent the container, the magnetic field strength detector configured to sense changes in the magnetic field as a result of movement of the bobber and magnet responsive to the oscillating motion of the container and clotting of the sample.

2. The apparatus of claim 1, wherein the container and the bobber each are formed as cylinders.

3. The apparatus of claim 2, comprising a space defined between an inner wall of the container and an outer surface of the bobber, the sample being disposed in the space.

4. The apparatus of claim 1, an aligning structure coupled between the container and the bobber, the aligning structure configured to align bobber within the container.

5. The apparatus of claim 4, wherein the aligning structure comprises a pintle formed in the container and a dimple formed on the bobber.

6. The apparatus of claim 5, wherein the aligning structure comprises an optional locating member coupled to the bobber.

7. The apparatus of claim 1, wherein the container or bobber is formed of medical grade plastic

8. The apparatus of claim 1, wherein the bobber is formed with a cavity into which the magnet is disposed.

9. The apparatus of claim 1, wherein the magnet is encapsulated within the bobber

10. The apparatus of claim 1, wherein the magnetic field generator comprises a permanent magnet disposed adjacent the container.

11. The apparatus of claim 1, wherein the magnetic field generator comprises first and second permanent magnets disposed adjacent the container.

12. The apparatus of claim 1, wherein the magnetic field generator provides a predetermined resistance to rotation of the bobber by interaction with the magnet.

13. The apparatus of claim 1, the drive assembly comprising a cam and motor arrangement, a direct motor drive or a gear drive

14. The apparatus of claim 1, container and bobber being configured to be replaceably inserted into the apparatus.

15. A container and bobber assembly for use with the apparatus of claim 1.

16. The apparatus of claim 1, the magnetic field strength detector configured to sense displacement of the bobber from an aligned position by sensing variations of the magnetic field.

17. The apparatus of claim 1, the magnetic field strength detector configured to provide a signal related to the strength of the sample undergoing clotting.

18. The apparatus of claim 17, the magnetic field strength detector coupled to a processing device.

19. The apparatus of claim 1, the magnetic field strength generator being configured to provide a magnetic field strength of a predetermined strength sufficient to at least partially restrain movement of the magnet and hence the bobber in view of a maximum clot strength of the sample.

20. The apparatus of claim 1, the magnetic field strength generator being configured to provide a magnetic field strength of a predetermined strength sufficiently low so as to permit movement of the magnet and hence the bobber substantially with initial clot formation.
